(19) United States
(12) Patent Application Publication
(10) Pub. No.: US 2024/0318251 A1
(43) Pub. Date: Sep. 26, 2024

(54) HIGH THROUGHPUT NEWBORN SCREENING ASSAY FOR ANGELMAN AND PRADER-WILLI SYNDROMES

(71) Applicant: Wisconsin Alumni Research Foundation, Madison, WI (US)

(72) Inventor: Mei Baker, Madison, WI (US)

(73) Assignee: Wisconsin Alumni Research Foundation, Madison, WI (US)

(21) Appl. No.: 18/612,463

(22) Filed: Mar. 21, 2024

Related U.S. Application Data
(60) Provisional application No. 63/491,564, filed on Mar. 22, 2023.

(51) Int. Cl. C12Q 1/6883; C12Q 1/6806

(52) U.S. Cl. CPC C12Q 1/6883; C12Q 1/6806; C12Q 2600/154; C12Q 2600/156

(57) ABSTRACT

Described herein is a method detecting a DNA methylation pattern in the small nuclear ribonucleoprotein polypeptide N (SNRPN) promoter region. Advantageously, the DNA is extracted from a dried blood sample from a human subject. In the method, treating the DNA with sodium bisulfite chemically modifies unmethylated cytosines in the DNA to uracil. The DNA is then amplified using specified forward and reverse primers for methylated and unmethylated SNRPN. The primer concentrations and amplification cycles are defined. A melting curve analysis of the PCR amplification products determines the DNA methylation pattern in the alleles in the DNA. The identified DNA methylation pattern can indicate normal status, Angelman syndrome or Prader-Willi syndrome.

### CROSS-REFERENCE TO RELATED APPLICATIONS

This application claims priority to U.S. Provisional Application No. 63/491,564 filed on Mar. 22, 2023, which is incorporated herein by reference in its entirety.

### SEQUENCE LISTING

The Instant Application contains a Sequence Listing which has been submitted electronically in XML format and is hereby incorporated by reference in its entirety. Said XML copy, created on Mar. 20, 2024, is named “SEQ_LIST—107668277-P230235US02.xml” and is 19,802 bytes in size. The Sequence Listing does not go beyond the disclosure in the application as filed.

### FIELD OF THE DISCLOSURE

The present disclosure is related to novel high throughput newborn screening assays for the detection of small nuclear ribonucleoprotein polypeptide N (SNRPN) promoter methylation status relevant to Angelman and Prader-Willi Syndrome.

### BACKGROUND

Angelman syndrome (AS) is a rare genetic disorder that primarily affects the nervous system. AS is characterized by severe developmental delay, learning disability, severe speech impairment, gait ataxia and/or tremulousness of the limbs. A unique behavior associated with AS is an apparent happy demeanor that includes frequent laughing, smiling, and excitability. Microcephaly and seizures are also common. Developmental delays are first noted at around age six months; however, the unique clinical features of AS do not become manifest until after age one year. AS is caused by loss of expression of the imprinted gene UBE3A, which in neurons is expressed from the maternal allele only. Lack of DNA methylation at the small nuclear ribonucleoprotein polypeptide N (SNRPN) promoter leads to the expression of a large SNRPN sense/UBE3A antisense transcript (SNHG14) on the maternal allele, which silences UBE3. Early detection may allow for early intervention such as gene therapy.

Prader-Willi Syndrome (PWS) is also a rare genetic disorder. In infants, PWS is characterized by poor muscle tone, distinctive facial features, and poor responsiveness. A key feature of PWS is a constant sense of hunger that usually begins at about two years of age. PWS is caused by the loss of function of the paternally inherited 15q11-q13 locus. The vast majority of PWS patients will show only methylated alleles at the small nuclear ribonucleoprotein polypeptide N (SNRPN) promoter. For reference, unaffected individuals will show one unmethylated allele and one methylated allele. Early detection may allow for early intervention such as gene therapy.

What is need are improved genetic screening methods for AS and PWS.

### BRIEF SUMMARY

In an aspect, a method of detecting a DNA methylation pattern comprises
providing DNA extracted from a dried blood sample from a human subject;
treating the DNA with sodium bisulfite to chemically modify unmethylated cytosines in the DNA to uracil;
amplifying the DNA in an amplification mixture comprising forward and reverse region primers for an unmethylated the small nuclear ribonucleoprotein polypeptide N (SNRPN) promoter region in amounts of 400 to 1000 nM, and forward and reverse region primers for a methylated SNRPN promoter region in amounts of 100 to 500 nM to provide PCR amplification products;
performing a melting curve analysis of the PCR amplification products; and
determining the DNA methylation pattern in the alleles in the DNA;
wherein the amplification mixture does not include dUTP or Uracil-DNA glycosylase,
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C.;
wherein the melting curve analysis comprises a cycle at a denaturing temperature of 95° C.-98° C., annealing at 56° C.-64° C., and re-denaturing at 95° C.-98° C.;
   wherein the unmethylated SNRPN promoter region forward primer comprises GTGTGGTTGTAGAGGTAGGTTGGTGT (SEQ ID NO: 6),
   the unmethylated SNRPN promoter region reverse primer comprises CAACTAACCTTACCCACTCCATCACA (SEQ ID NO: 7),
   the methylated SNRPN promoter region forward primer comprises CGGTCGTAGAGGTAGGTTGGCGC (SEQ ID NO: 8), and
   the methylated SNRPN promoter region reverse primer comprises CAACTAACCTTACCCACTCCATCACA (SEQ ID NO: 9).

### BRIEF DESCRIPTION OF THE DRAWINGS

FIG. 1 is a schematic of the assay. CTGCGCGGCCGCAGAG (SEQ ID NO: 1) is the allele sequence prior to bisulfite treatment; UTGCGCGGUCGUAGAG (SEQ ID NO: 2) is the sequence of the methylated allele after bisulfite treatment; UTGUGUGGUUGUAGAG (SEQ ID NO: 3) is the sequence of the unmethylated allele after bisulfite treatment; TTGCGCGGTCGTAGAG (SEQ ID NO: 4) is the sequence of the methylated allele after RT-PCR; and TTGTGTGGTTGTAGAG (SEQ ID NO: 5) is the sequence of the unmethylated allele after RT-PCR.

FIG. 2 shows the results of a melting curve analysis.

FIG. 3 shows the agarose gel analysis of the 152-bp and 92-bp fragments amplified by verification primers SEQ ID NOs: 10-13.

The above-described and other features will be appreciated and understood by those skilled in the art from the following detailed description, drawings, and appended claims.

### DETAILED DESCRIPTION

Described herein is a high throughput newborn screening assay for the detection of small nuclear ribonucleoprotein polypeptide N (SNRPN) promoter methylation status relevant to AS and PWS. The assay principle is to assess the small nuclear ribonucleoprotein polypeptide N (SNRPN) promoter methylation status using methylation-specific PCR coupled with a melting curve analysis to identify AS caused by deletion of the maternal allele, paternal uniparental disomy, and imprinting center defects, and PWS caused by deletion of the paternal allele, maternal uniparental disomy, and imprinting center defects. It has been unexpectedly found that the standard methods described in the art for methylation-specific PCR coupled with a melting curve analysis cannot reliably detect SNRPN promoter methylation. Advantageously, the method is adapted for high throughput screening in a 96-well plate setting, for example. The methods described herein thus fulfill this need.

A method of detecting a DNA methylation pattern comprises
providing DNA extracted from a dried blood sample from a human subject;
treating the DNA with sodium bisulfite to chemically modify unmethylated cytosines in the DNA to uracil;
amplifying the DNA in an amplification mixture comprising forward and reverse region primers for an unmethylated small nuclear ribonucleoprotein polypeptide N (SNRPN) promoter region in amounts of 400 to 1000 nM, and forward and reverse region primers for a methylated SNRPN promoter region in amounts of 100 to 500 nM to provide PCR amplification products;
performing a melting curve analysis of the PCR amplification products; and
determining the DNA methylation pattern in the alleles in the DNA;
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C.;
wherein the melting curve analysis comprises a cycle at a denaturing temperature of 95° C.-98° C., annealing at 56° C.-64° C., and re-denaturing at 95° C.-98° C.; and
   wherein the unmethylated SNRPN promoter region forward primer comprises GTGTGGTTGTAGAGGTAGGTTGGTGT (SEQ ID NO: 6),
   the unmethylated SNRPN promoter region reverse primer comprises CAACTAACCTTACCCACTCCATCACA (SEQ ID NO: 7),
   the methylated SNRPN promoter region forward primer comprises CGGTCGTAGAGGTAGGTTGGCGC (SEQ ID NO: 8), and
   the methylated SNRPN promoter region reverse primer comprises CAACTAACCTTACCCACTCCATCACA (SEQ ID NO: 9).

In an aspect, the human subject is a human newborn. The assay described herein can be integrated into routine newborn genetic screening.

Advantageously, the methods described herein can be performed in a high throughput or multi-well format, such as a 96 well format.

The DNA used in the method is extracted from a dried blood sample. Dried blood samples can be prepared by using a sterile lancet to pierce the skin of the test recipient, typically on a fingertip or a heel of a newborn, where a blood droplet is allowed to briefly collect on the patient's skin before being spotted on Grade 903 Whatman™ filter paper or equivalent. Saturation of the filter paper is required, where an approximate 60 μL of blood formed circle of spot is visible on each side of the collection paper. To complete the dried blood spot creation process, the collected sample is dried at room temperature for a minimum of three hours. Dried spots may be stored up to 2-4 weeks at room temperature, up to one year at 4° C. or up to multiple years at −20° C. or −80° C.

To perform the analysis, typically circular punches ranging in diameters from about 1/32^{nd} to ¼^{th} inch are made which contain a known amount of the original blood product volume. A typical punch diameter taken from dried blood spots is ⅛^{th} inch that contains 3.2 μL of whole blood.

The DNA can be extracted from the dried blood sample using methods known in the art. An exemplary method is described in U.S. Pat. No. 9,206,468, incorporated herein by reference for its disclosure of DNA extraction methods. For example, a one-step elution method comprises adding a one-step elution buffer to the blood sample to form a mixture, wherein the one-step elution buffer consists of 5 to 22.5 mM potassium in the form of KOH, KCl, or a combination thereof, and 7.5 to 30 mM of a base having a buffering range of 7.0 to 9.5, wherein the pH of the one-step elution buffer is 9 to 13, heating the mixture at 90° C. to 99° C. for a time sufficient to elute the DNA from the blood sample to form an eluted DNA solution, and optionally cooling the eluted DNA solution at a temperature of at least 4° C. for at least 5 minutes, wherein the eluted DNA solution is suitable for direct use in an enzymatic DNA amplification reaction. An exemplary commercially available product is Extracta™ DBS, available from Quantabio.

Advantageously, the DNA is a portion of a DNA sample extracted for testing for severe combined immunodeficiency and spinal muscular atrophy newborn screening. Thus, the methods described herein can be readily integrated into newborn screening programs in public health laboratories throughout the United States, which potentially impacts 4 million newborns born in the United States each year.

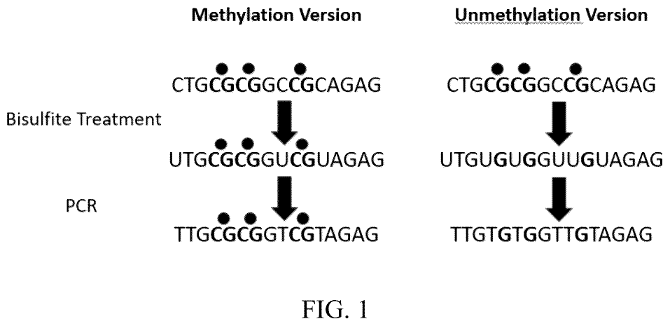

The DNA is then treated with sodium bisulfite to chemically modify unmethylated cytosines in the DNA to uracil. Sodium bisulfite treatment can comprise using a kit known in the art such as EZ-96 DNA Methylation-Gold™ Kit. Exemplary conditions include treating DNA with 3-8 M sodium bisulfite for 100 to 200 minutes at 56 to 68° C. after DNA samples are denatured at 95-98° C. for 5-10 minutes, to convert all unmethylated cytosine residues in the DNA to uracil. FIG. 1 illustrates the results of bisulfite treatment.

After the sodium bisulfite treatment, the DNA is amplified. DNA amplification mixtures are well-known in the art and include a thermostable DNA polymerase, buffers and dNTPs. An exemplary commercial kit is 1× Quanta Multiplex Toughmix®.

In the amplification step, the forward and reverse region primers for an unmethylated small nuclear ribonucleoprotein polypeptide N (SNRPN) promoter region are in amounts of 400 to 1000 nM, and forward and reverse region primers for a methylated SNRPN promoter region are in amounts of 100 to 500 nM to provide PCR amplification products. The inventor found that these primer concentrations are critical to detect both the methylated and unmethylated alleles and provide accurate results.

In an aspect, in the amplification step, the forward and reverse region primers for the unmethylated SNRPN promoter region are both in amounts of 1000 nM, and the forward and reverse region primers for the methylated SNRPN promoter region are in amounts of 100 nM and 500 nM, respectively.

The amplification is done using one cycle at an initial denaturing temperature of 95° C.-98° C. (cycle time 5 to 15 minutes, preferably 5 to 10 minutes), followed by 30-42 denaturing/annealing/extension cycles at a denaturing temperature of 95° C.-98° C. (5 to 30 seconds, preferably 10 to 20 seconds), an annealing temperature of 56° C.-64° C. (10 to 60 seconds, preferably 20 to 40 seconds), and an extension temperature of 68° C.-76° C. (20 to 90 seconds, preferably 20 to 60 seconds). The inventor has found that using a denaturing temperature of 95° C.-98° C. rather than the more typical 94° C. improves the detection of both the methylated and unmethylated alleles and provides accurate results.

Advantageously, because of the DNA methylation step, the amplification mixture does not include dUTP or Uracil-DNA glycosylase.

After amplification, a melting curve analysis is performed. The melting curve analysis comprises a cycle at a denaturing temperature of 95° C.-98° C., annealing at 56° C.-64° C., and re-denaturing at 95° C.-98° C. The results of the melting curve analysis allow one to identify the methylated and unmethylated alleles as the methylated and unmethylated alleles have different melting temperatures.

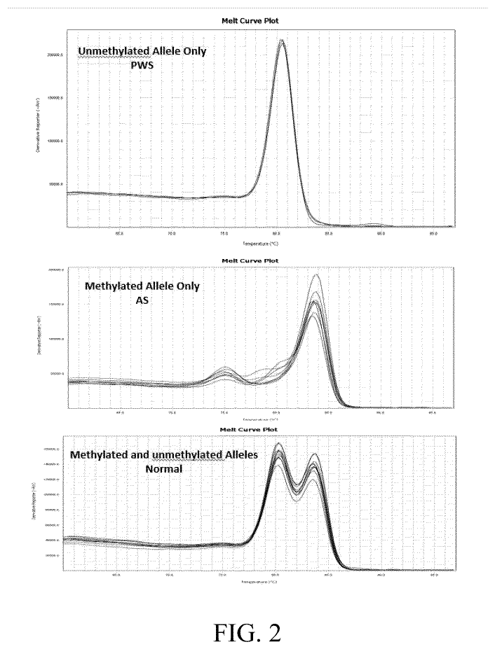

The results of the melting curve analysis are interpreted as follows and as shown in FIG. 2:
both methylated and unmethylated alleles in the DNA indicates a normal DNA sample;
only unmethylated alleles in the DNA indicates Angelman syndrome; and
only methylated alleles in the DNA indicates Prader-Willi syndrome.

In an aspect, in order to confirm a positive result obtained in the melting curve analysis, a second methylation-sensitive amplification can be performed with alternative primers SEQ ID Nos. 10-13. In the second methylation-sensitive amplification, the alternative forward and reverse region primers for an unmethylated small nuclear ribonucleoprotein polypeptide N (SNRPN) promoter region in amounts of 400 to 1000 nM, and the alternative forward and reverse region primers for a methylated SNRPN promoter region in amounts of 400 to 1000 nM to provide PCR amplification products. The amplification is done using one cycle at an initial denaturing temperature of 95° C.-98° C. (cycle time 5 to 15 minutes, preferably 5 to 10 minutes), followed by 30-42 denaturing/annealing/extension cycles at a denaturing temperature of 95° C.-98° C. (5 to 30 seconds, preferably 20 to 30 seconds), an annealing temperature of 56° C.-64° C. (10 to 60 seconds, preferably 20 to 30 seconds), and an extension temperature of 68° C.-76° C. (20 to 90 seconds, preferably 30 to 40 seconds).

The alternative primers are as follows:
the alternative methylated SNRPN promoter region forward primer comprises TCGATGGTATTTTGTTCGTTCGTATTGGGGCGC (SEQ ID NO: 10),
the alternative methylated SNRPN promoter region reverse primer comprises CCATATCCCTTACCCACTACGTTTACCCCG (SEQ ID NO: 11),
the alternative unmethylated SNRPN promoter region forward primer comprises TTGATGGTATTTTGTTTGTTTGTATTGGGGTGT (SEQ ID NO: 12), and
the alternative unmethylated SNRPN promoter region reverse primer comprises ACCACAGACACCCACAATAAAACCTATCACA (SEQ ID NO: 13)

Advantageously, if the subject, particularly a human newborn, is indicated to have either Angelman syndrome or Prader-Willi syndrome, intervention can be started even before the subject's symptoms become pronounced.

In an aspect, a determination of only unmethylated allele in the DNA indicates Angelman syndrome. The initial screening, for example high throughput screening using the primers of SEQ ID NOs. 6-9, can be subjected to further verification when Angelman syndrome is suspected.

In an aspect, the method further comprises
amplifying the chemically modified DNA in an amplification mixture comprising alternative forward and reverse region primers for an unmethylated small nuclear ribonucleoprotein polypeptide N (SNRPN) promoter region in amounts of 400-1000 nM, and alternative forward and reverse region primers for a methylated SNRPN promoter region in amounts of 400-1000 nM; and
performing a gel electrophoresis of the PCR amplification products with the forward and reverse region primers for unmethylated and methylated SNRPN; and
visualizing, in the gel electrophoresis, only 92-bp fragments for the unmethylated allele to confirm Angelman syndrome, or visualizing, in the gel electrophoresis, a 152-bp fragment for the methylated to confirm no Angelman syndrome.
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C., and
   wherein the alternative methylated SNRPN promoter region forward primer comprises TCGATGGTATTTTGTTCGTTCGTATTGGGGCGC (SEQ ID NO: 10),
   the alternative methylated SNRPN promoter region reverse primer comprises CCATATCCCTTACCCACTACGTTTACCCCG (SEQ ID NO: 11),
   the alternative unmethylated SNRPN promoter region forward primer comprises TTGATGGTATTTTGTTTGTTTGTATTGGGGTGT (SEQ ID NO: 12), and
the alternative unmethylated SNRPN promoter region reverse primer comprises ACCACAGACACCCACAATAAAACCTATCACA (SEQ ID NO: 13).

In an aspect, a determination of only methylated allele in the DNA indicates Prader-Willi syndrome. The initial screening, for example high throughput screening using the primers of SEQ ID NOs. 6-9, can be subjected to further verification when Prader-Willi Syndrome is suspected.

In an aspect, the method further comprises
amplifying the chemically modified DNA in an amplification mixture comprising alternative forward and reverse region primers for an unmethylated small nuclear ribonucleoprotein polypeptide N (SNRPN) promoter region in amounts of 400-1000 nM, and alternative forward and reverse region primers for a methylated SNRPN promoter region in amounts of 400-1000 nM; and
performing a gel electrophoresis of the PCR amplification products with the forward and reverse region primers for unmethylated and methylated SNRPN; and
visualizing, in the gel electrophoresis, only 152-bp fragments for the methylated allele to confirm Prader-Willi syndrome, or visualizing, in the gel electrophoresis, a 92-bp fragment for the methylated allele to confirm no Prader-Willi syndrome,
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C., and
   wherein the alternative methylated SNRPN promoter region forward primer comprises TCGATGGTATTTTGTTCGTTCGTATTGGGGCGC (SEQ ID NO: 10),
   the alternative methylated SNRPN promoter region reverse primer comprises CCATATCCCTTACCCACTACGTTTACCCCG (SEQ ID NO: 11),
   the alternative unmethylated SNRPN promoter region forward primer comprises TTGATGGTATTTTGTTTGTTTGTATTGGGGTGT (SEQ ID NO: 12), and
   the alternative unmethylated SNRPN promoter region reverse primer comprises ACCACAGACACCCACAATAAAACCTATCACA (SEQ ID NO: 13).

Treatments for AS include administration of anti-seizure medication, physical therapy, speech therapy, occupational therapy, behavioral therapy, and combinations thereof. Gene therapy can include insertion of a therapeutic UBE3A gene.

Treatments for PWS include human growth hormone treatment, nutritional intervention, sex hormone treatment, weight management, physical therapy, speech therapy, occupational therapy, behavioral therapy, and combinations thereof.

### EXAMPLES

### Example 1: DNA Isolation and Sodium Bisulfite Treatment of DNA

DNA was isolated from routine newborn screening (NBS) dried blood spot specimens using Extracta DBS™. Each 3.2 mm DBS punch was eluted in 54 μL of Extracta™ DBS with incubation at 96° C. for 25 minutes after it was washed once with 90 μL of the same solution. For the sodium bisulfite treatment of DNA, the EZ-96 DNA Methylation-Gold™ Kit was used to treat 40 μL of DNA isolated solution. The process included mixing the DNA samples with the conversion reagent in a 96-well plate setting, then the mixture underwent incubation in a thermal cycler with 98° C. for 10 minutes and 64° C. for 150 minutes followed by the DNA purification using spin columns provided in the kit. This process results in the conversion of unmethylated cytosine to uracil, without changing the status of methylated cytosine.

All of the newborn screening programs in the United States screen for severe combined immunodeficiency that use a DNA based assay, so the same DNA isolation products can also be used for the described AS/PWS screening assay.

### Example 2: Primer Set 1: PCR Reaction and Melting Curve Analysis

PRIMER SET 1 targets the sequence CTGCGCGGCCGCAGAG (SEQ ID NO: 1). The 2-10 μL of sodium bisulfite treated DNA was subjected to a PCR amplification reaction. The PCR reaction mixture included a primer set that targets the unmethylated promoter region of SNRPN: Forward-5′ GTGTGGTTGTAGAGGTAGGTTGGTGT 3′ (SEQ ID NO: 6); Reverse-5′ CAACTAACCTTACCCACTCCATCACA 3′ (SEQ ID NO: 7), and another set of primer that targets the methylated promoter region of SNRPN: Forward-5′ CGGTCGTAGAGGTAGGTTGGCGC 3′ (SEQ ID NO: 8); Reverse-5′ CAACTAACCTTACCCACTCCATCACA 3′ (SEQ ID NO: 9). The concentration of the unmethylated primer set was 400 nM to 1000 nM, and the concentration of methylated primer set was 125 nM to 500 nM. A total of 20 μL reaction mixture also contained 1× Quanta Multiplex Toughmix® that does not include dUTP or Uracil-DNA glycosylase, and 1× dsGreen dye. The PCR conditions were 94° C. to 98° C. for 5 minutes followed by 40 cycles of denaturation at 94° C. to 98° C. for 10-20 seconds, annealing at 63° C. to 71° C. for 20 to 40 seconds, and extension at 68° C. to 74° C. for 20 to 60 seconds. An additional cycle was followed with denaturation at 95° C. to 99° C. for 15 seconds, annealing at 58° C. to 62° C. for 60 seconds, and denaturation again at 95° C. to 99° C. for 15 seconds.

### Example 3: Exemplary Assay Using Primer Set 1 and Results

The following conditions were used:

In a 20 μL reaction mixture:
Forward unmethylated primer—1000 nM
Reverse unmethylated primer—1000 nM
Forward methylated primer—250 nM
Reverse methylated primer—250 nM
dsGreen Dye—1×
Quanta ToughMix™—1×
Sodium Bisulfate Treated DNA—6 μL

Thermal Cycler Condition:
1. 97° C. for 5 min
2. 97° C. for 10 sec
3. 60° C. for 30 sec
4. 72° C. for 40 sec
5. Repeat 2-4 for 40 cycles
6. 97° C. for 15 sec
7. 60° C. for 60 sec
8. 97° C. for 15 sec

The results of a typical 96-well assay are shown in FIG. 2. As shown in FIG. 2, the method can identify the AS and PWS status of newborns.

### Example 4: Primer Set 2: PCR Reaction and Melting Curve Analysis

Based on a report by Askree et al (Hussain Askree S, Hjelm L N, Ali Pervaiz M, Adam M. Bean L J, Hedge M, Coffee B. Allelic dropout can cause false-positive results for Prader-Willi and Angelman syndrome testing. J Mol Diagn. 2011 January; 13(1):108-12. doi: 10.1016/j.jmoldx.2010.11.006. Epub 2010 Dec. 23. PMID: 21227401; PMCID: PMC3069869), an alternate methylation sensitive PCR assay was designed using two different primer sets to confirm screening positive results from the described high throughput newborn screening assay for Angelman and Prader-Willi Syndrome, ruling out the possibility of false positive results due to allelic dropout (ADO). ADO involves selective allele amplification during PCR thermocycling. The presence of single nucleotide variants (SNVs) in the forward and/or reverse primer binding sites may lead to the complete or partial lack of amplification of the single allele, while the second one may “drop” out during the PCR process. Thus ADO can reduce the efficiency of PCR-based sequencing. The primers of Hussain Askree et al. were thus used in the methods described herein.

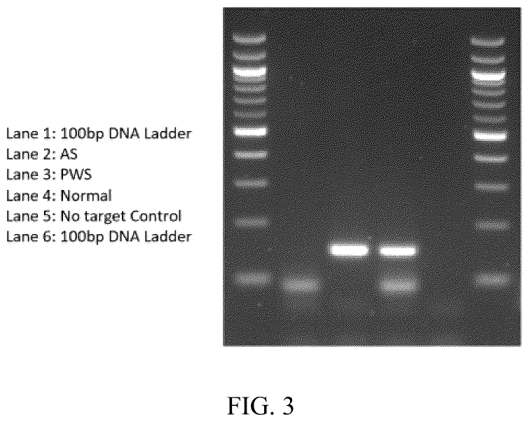

In a total of 25 μL, the reaction mixture contained 1×PCR buffer, each primer at 1 μM, dNTPs at 200 μM (excluding dUTP), MgCl₂ at 2.5 mM, and 4 μL of sodium bisulfite treated DNA. The PCR conditions were 95° C. for 5 minutes followed by 35 cycles of denaturation at 95° C. for 30 seconds, annealing at 64° C. for 30 seconds, and extension at 72° C. for 40 seconds. PRIMSER SET 2 includes: Alt-MF (5′-TCGATGGTATTTTGTTCGTTCGTATTGGGGCGC-3′; SEQ ID NO: 10), Alt-MR (5′-CCATATCCCTTACCCACTACGTTTACCCCG-3′; SEQ ID NO: 11), Alt-PF (5′-TTGATGGTATTTTGTTTGTTTGTATTGGGGTGT-3′; SEQ ID NO: 12), and Alt-PR (5′-ACCACAGACACCCACAATAAAACCTATCACA-3′; SEQ ID NO: 13). The alternate primers amplify a 152-bp fragment from the methylated allele and a 92-bp fragment from the unmethylated allele, and are visualized using agarose gel electrophoresis (FIG. 3). Under the improved methylation-specific PCR conditions described herein, PRIMER SET 2 can be used as an alternative to PRIMER SET 1 to detect SNRPN promoter methylation status relevant to AS and PWS.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of detecting a DNA methylation pattern, comprising
providing DNA extracted from a dried blood sample from a human subject;
treating the DNA with sodium bisulfite to chemically modify unmethylated cytosines in the DNA to uracil;
amplifying the DNA in an amplification mixture comprising forward and reverse region primers for an unmethylated small nuclear ribonucleoprotein polypeptide N (SNRPN) promoter region in amounts of 400 to 1000 nM, and forward and reverse region primers for a methylated SNRPN promoter region in amounts of 100 to 500 nM; and
performing a melting curve analysis of the PCR amplification products and determining the DNA methylation pattern in the alleles in the DNA;
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C.,
wherein the melting curve analysis comprises a cycle at a denaturing temperature of 95° C.-98° C., annealing at 56° C.-64° C., and re-denaturing at 95° C.-98° C., and
the unmethylated SNRPN promoter region forward primer comprises GTGTGGTTGTAGAGGTAGGTTGGTGT (SEQ ID NO: 6),
the unmethylated SNRPN promoter region reverse primer comprises CAACTAACCTTACCCACTCCATCACA (SEQ ID NO: 7),
the methylated SNRPN promoter region forward primer comprises CGGTCGTAGAGGTAGGTTGGCGC (SEQ ID NO: 8), and
the methylated SNRPN promoter region reverse primer comprises CAACTAACCTTACCCACTCCATCACA (SEQ ID NO: 9).

2. The method of claim 1, further comprising extracting the DNA from the dried blood sample.

3. The method of claim 1, wherein the DNA is a portion of a DNA sample extracted for severe combined immunodeficiency and spinal muscular atrophy newborn screening.

4. The method of claim 1, the forward and reverse region primers for the unmethylated SNRPN promoter region are both in amounts of 1000 nM, and the forward and reverse region primers for the methylated SNRPN promoter region are in amounts of 100 nM and 500 nM, respectively.

5. The method of claim 1, wherein the method is performed as a high throughput assay.

6. The method of claim 5, wherein the assay is performed in 96 well format.

7. The method of claim 1, wherein the human subject is human newborn.

8. The method of claim 1, wherein a determination of both methylated and unmethylated alleles in the DNA indicates a normal DNA sample.

9. The method of claim 1, wherein a determination of only unmethylated alleles in the DNA indicates Angelman syndrome.

10. The method of claim 9, further comprising
amplifying the chemically modified DNA in an amplification mixture comprising alternative forward and reverse region primers for an unmethylated small nuclear ribonucleoprotein polypeptide N (SNRPN) promoter region in amounts of 400-1000 nM, and alternative forward and reverse region primers for a methylated SNRPN promoter region in amounts of 400-1000 nM; and
performing a gel electrophoresis of the PCR amplification products with the forward and reverse region primers for unmethylated and methylated SNRPN; and
visualizing, in the gel electrophoresis, only 92-bp fragments for the unmethylated allele to confirm Angelman syndrome, or visualizing, in the gel electrophoresis, a 152-bp fragment for the methylated allele to confirm no Angelman syndrome,
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C., and
wherein the alternative methylated SNRPN promoter region forward primer comprises TCGATGGTATTTTGTTCGTTCGTATTGGGGCGC (SEQ ID NO: 10),
the alternative methylated SNRPN promoter region reverse primer comprises CCATATCCCTTACCCACTACGTTTACCCCG (SEQ ID NO: 11),
the alternative unmethylated SNRPN promoter region forward primer comprises TTGATGGTATTTTGTTTGTTTGTATTGGGGTGT (SEQ ID NO: 12), and
the alternative unmethylated SNRPN promoter region reverse primer comprises ACCACAGACACCCACAATAAAACCTATCACA (SEQ ID NO: 13).

11. The method of claim 1, wherein a determination of only methylated alleles in the DNA indicates Prader-Willi syndrome.

12. The method of claim 11, further comprising
amplifying the chemically modified DNA in an amplification mixture comprising alternative forward and reverse region primers for an unmethylated small nuclear ribonucleoprotein polypeptide N (SNRPN) promoter region in amounts of 400-1000 nM, and alternative forward and reverse region primers for a methylated SNRPN promoter region in amounts of 400-1000 nM; and
performing a gel electrophoresis of the PCR amplification products with the forward and reverse region primers for unmethylated and methylated SNRPN; and
visualizing, in the gel electrophoresis, only 152-bp fragments for the methylated allele to confirm Prader-Willi syndrome, or visualizing, in the gel electrophoresis, a 92-bp fragment for the unmethylated allele to confirm no Prader-Willi syndrome,
wherein the amplification mixture does not include dUTP or Uracil-DNA glycosylase;
wherein the amplifying is done using one cycle at an initial denaturing temperature of 95° C.-98° C., followed by 30-42 denaturing/annealing/extension cycles at a denaturing temperature of 95° C.-98° C., an annealing temperature of 56° C.-64° C., and an extension temperature of 68° C.-76° C., and
wherein the alternative methylated SNRPN promoter region forward primer comprises TCGATGGTATTTTGTTCGTTCGTATTGGGGCGC (SEQ ID NO: 10),
the alternative methylated SNRPN promoter region reverse primer comprises CCATATCCCTTACCCACTACGTTTACCCCG (SEQ ID NO: 11),
the alternative unmethylated SNRPN promoter region forward primer comprises TTGATGGTATTTTGTTTGTTTGTATTGGGGTGT (SEQ ID NO: 12), and
the alternative unmethylated SNRPN promoter region reverse primer comprises ACCACAGACACCCACAATAAAACCTATCACA (SEQ ID NO: 13).

13. A method of treating a human subject, comprising performing the assay of claim 1, determining only unmethylated alleles in the DNA, and treating the human subject for Angelman syndrome.

14. A method of treating a human subject, comprising performing the assay of claim 1, determining only methylated alleles in the DNA, and treating the human subject for Prader-Willi syndrome.

15. A method of treating a human subject, comprising performing the assay of claim 10, determining only unmethylated alleles in the DNA, and treating the human subject for Angelman syndrome.

16. A method of treating a human subject, comprising performing the assay of claim 12, determining only methylated alleles in the DNA, and treating the human subject for Prader-Willi syndrome.


SEQUENCE LISTING (13 sequences)

SEQ ID NO: 1 - DNA, 16 bp, Homo sapiens
  source 1..16: mol_type = genomic DNA
ctgcgcggccgcagag

SEQ ID NO: 2 - DNA, 16 bp, synthetic construct
  source 1..16: mol_type = other DNA; note = Methylated allele after bisulfite treatment
  modified_base 1: mod_base = OTHER; note = uracil
  modified_base 9: mod_base = OTHER; note = uracil
  modified_base 12: mod_base = OTHER; note = uracil
ctgcgcggccgcagag

SEQ ID NO: 3 - DNA, 16 bp, synthetic construct
  source 1..16: mol_type = other DNA; note = Unmethylated allele after bisulfite treatment
  modified_base 1: mod_base = OTHER; note = uracil
  modified_base 4: mod_base = OTHER; note = uracil
  modified_base 6: mod_base = OTHER; note = uracil
  modified_base 9: mod_base = OTHER; note = uracil
  modified_base 10: mod_base = OTHER; note = uracil
  modified_base 12: mod_base = OTHER; note = uracil
ctgcgcggccgcagag

SEQ ID NO: 4 - DNA, 16 bp, synthetic construct
  source 1..16: mol_type = other DNA; note = Methylated allele after RT-PCR
ttgcgcggtcgtagag

SEQ ID NO: 5 - DNA, 16 bp, synthetic construct
  source 1..16: mol_type = other DNA; note = Unmethylated allele after RT-PCR
ttgtgtggttgtagag

SEQ ID NO: 6 - DNA, 26 bp, synthetic construct
  source 1..26: mol_type = other DNA; note = Unmethylated SNRPN promoter region forward primer
gtgtggttgtagaggtaggttggtgt

SEQ ID NO: 7 - DNA, 26 bp, synthetic construct
  source 1..26: mol_type = other DNA; note = Unmethylated SNRPN promoter region reverse primer
caactaaccttacccactccatcaca

SEQ ID NO: 8 - DNA, 23 bp, synthetic construct
  source 1..23: mol_type = other DNA; note = Methylated SNRPN promoter region forward primer
cggtcgtagaggtaggttggcgc

SEQ ID NO: 9 - DNA, 26 bp, synthetic construct
  source 1..26: mol_type = other DNA; note = Methylated SNRPN promoter region reverse primer
caactaaccttacccactccatcaca

SEQ ID NO: 10 - DNA, 33 bp, synthetic construct
  source 1..33: mol_type = other DNA; note = Methylated SNRPN promoter region forward primer
tcgatggtattttgttcgttcgtattggggcgc

SEQ ID NO: 11 - DNA, 30 bp, synthetic construct
  source 1..30: mol_type = other DNA; note = Methylated SNRPN promoter region reverse primer
ccatatcccttacccactacgtttaccccg

SEQ ID NO: 12 - DNA, 33 bp, synthetic construct
  source 1..33: mol_type = other DNA; note = Unmethylated SNRPN promoter region forward primer
ttgatggtattttgtttgtttgtattggggtgt

SEQ ID NO: 13 - DNA, 31 bp, synthetic construct
  source 1..31: mol_type = other DNA; note = Unmethylated SNRPN promoter region reverse primer
accacagacacccacaataaaacctatcaca